(19) United States
(12) Patent Application Publication
(10) Pub. No.: US 2025/0248600 A1
(43) Pub. Date: Aug. 7, 2025

(54) Radiation-free, non-invasive, contact-less and cost-effective routine breast cancer screening device Brexwel for identifying abnormalities in female subjects

(71) Applicants: Deepika Singh, Prayagraj (IN); Gaurav Singh, Prayagraj (IN)

(72) Inventors: Deepika Singh, Prayagraj (IN); Gaurav Singh, Prayagraj (IN)

(21) Appl. No.: 19/190,641

(22) Filed: Apr. 27, 2025

(30) Foreign Application Priority Data
Apr. 28, 2024 (IN) 202411033706

(51) Int. Cl. A61B 5/00; A61B 5/01; G16H 10/60; G16H 15/00; G16H 20/00; G16H 50/30; G16H 70/00

(52) U.S. Cl. CPC A61B 5/0091; A61B 5/015; A61B 5/7267; G16H 10/60; G16H 15/00; G16H 20/00; G16H 50/30; G16H 70/00

(57) ABSTRACT

The device Brexwel based on photoplethysmography (PPG) is used to track changes in oxygen consumption in breast tissues which can detect early indicators of breast cancer. In order to ensure high sensitivity, good noise performance, simplicity, and the reduction of artefacts in PPG signals, the device has an accelerometer. Hot-spot detection is done using the Brexwel device's thermal camera. For multi-modal classification, sensor-based, thermal image-based, and patient medical history-based classification, the device is controlled by a mobile application. Through specially designed capabilities in the mobile application, the device identifies various breast problems based on certain thresholds from PPG sensor. It is possible to get above 80% accuracy and 90% sensitivity, according to the detailed analysis of multi-modal findings, including thermal, optical, and medical histories conducted on female subjects of various age groups under normal, benign and malignant category.

### CROSS-REFERENCE TO RELATED APPLICATIONS

The present application hereby claims priority to and incorporates by reference the entirety of the disclosure of the Indian patent application No. 202411033706, filed on 28 Apr. 2024.

### FIELD

Present invention relates to a medical device for monitoring female breast health by addressing the lack of easily accessible non-invasive low-cost device for monitoring breast health.

### BACKGROUND

Female breast cancer [H. Sung et al., “Global Cancer Statistics 2020: GLOBOCAN Estimates of Incidence and Mortality Worldwide for 36 Cancers in 185 Countries,” CA. Cancer J. Clin., vol. 71, no. 3, pp. 209-249, 2021, doi: 10.3322/caac.21660] has surpassed lung cancer as the most commonly diagnosed cancer worldwide and in India. In India, less than 5% of women's breast is screened per year due to limited medical and diagnostic facilities. 75% of female population with breast cancer is asymptomatic 1.e around 1.5 Lakh. There is a lack of easily assessable non-invasive low-cost routine breast cancer screening device in diagnostic centres and health care centres in India. Existing gold standard mammography has following limitations: (1) Increased chances of repeated exposure (2) Painful (3) Being less effective for young women with dense breast. There is also the lack of quality control measure in mammography centers [1]. Also, there is a delay in report generation, especially in Tier 2 and Tier 3 cities in India. The machines used for mammography, Magnetic Resonance Imaging (MRI) and Ultrasound are non-portable and costly. The cancerous cells generate heat due to the following reasons: (1) Release of nitric oxide into the blood causing alteration in micro-circulation (2) Vasodilation: dilation of blood vessels to increase blood circulation (3) Neo-angiogenesis: Creation of new blood vessels to supply nutrients to tumor (4) Increase in metabolic activity of cancerous cells [D. Singh and A. K. Singh, “Role of image thermography in early breast cancer detection—Past, present and future,” Comput. Methods Programs Biomed., vol. 183, 2020, doi: 10.1016/j.cmpb.2019.105074].

Optical spectroscopy [2], [3] is considered as a real-time, quantitative, and less-invasive technique for optical characterization of biological samples. The main light absorber in tissues is hemoglobin in the whole optical spectrum. Total hemoglobin concentration reflects the degree of vascularization, which can be helpful in examining angiogenesis during cancer development. The absorption spectrum of hemoglobin can change with its degree of oxygenation. The Near Infra-red optical window from 750 nm to 950 nm can penetrate deeply into the breast tissues as the common minimum absorption trough of the major tissue components (water, deoxygenated and oxygenated blood, melanin etc, lies within this bandwidth. Raman spectroscopy based clinical studies measured calcification composition through varying thickness of tissues (2-10 mm). Thickness of skin in breast tissue in normal breast is 1.5 mm and in malignant breast, can be up to 5.00 mm. With 800 nm, the NIR can penetrate 3.56+/−0.34 mm skin depth. Existing NIR (NIROS) based techniques are costly and not commonly used in diagnostic centers or primary health centers in cities with limited medical facilities. The invention uses the principal of photoplethysmography to non-invasively measure the signals from breast tissues to indicate abnormality in tissues.

PPG sensors use simpler hardware implementation and have lower costs, and for operation, only a single sensor is required to be placed on the body.

PPG can be used to measure HRV, or the variations between heartbeat time intervals (Peak-to-Peak or P-P Interval) variation can be due to many factors such as the individual's age, heart conditions, and physical fitness. Factors affecting HRV include, but are not limited to, age, cancer and thermoregulation.

There is a need to develop a portable, radiation-free, non-invasive and cost-effective sensor based on optical imaging to reduce the mortality associated with late-stage diagnosis of breast cancer. The invention Brexwel includes usage of Photoplethysmography (PPG) based device to monitor change in oxygen consumption in breast tissues for identifying early signs of breast cancer. PPG is an uncomplicated and expensive optical measurement method that can be used to measure volumetric variations of blood circulation by measuring Heart Rate Variability (HRV), SpO2 and Pulse rate. The second derivative of the original PPG signal called the acceleration photoplethysmogram (APG) helps in identifying critical points a,—early systolic location, Point b—lowest point in the early systolic wave, point c—is the resurgent of the late systolic, Point d indicates the decreasing part of the late systolic and Point e represents the early diastolic wave. PPG signal waveform can also be used to measure atrial fibrillation [4] found sometimes in breast cancer patients. The device contains a accelerometer to improve the performance of device by ensuring high sensitivity, good noise performance, simplicity and reducing artifacts in PPG signals. The thermal camera in device will be used for placement of hot-spot identification. The device is operated by a mobile application for multi-modal classification, sensor-based, thermal image based and patient's medical history based. The device is also used for monitoring chemotherapy response by customized features in mobile application.

### SUMMARY

The present study aims to present the details of Brexwel device having following features:
1. Low-cost, easy-to-use, portable, radiation-free and non-invasive invention will be used in diagnostic centers, pathology centers, gynecology centers to perform following functions:
2. Correctly identify female subjects with breast problems like micro-calcification, neo-plastic etiology, etc. that may lead to cancer.
3. Implement user authentication and authorization mechanisms to ensure data security and privacy.
4. Create modules for uploading multi-modal data-sensor data, thermal images, running Machine Learning algorithms for analysis, and generating reports.
5. Use a mobile application for patients to access their reports, educational resources on breast cancer, and locate nearby diagnostic centers.
6. Collect and preprocess a dataset of thermal and optical data for training purposes.
7. Train a machine learning model to classify thermal images and identify potential signs of breast cancer.
8. Optimize the model for accuracy, sensitivity, and specificity
9. Personalized features for recommendation and follow-up
10. Good performance in low-frequency range and noise
11. Suitable for diagnostic centers/health centers in rural areas and Tier 2 and Tier 3 cities with limited medical facilities
12. Multistep data security
13. Easy-to-use graphic-user-interface and mobile application having multi-user management
14. Multi-modal classification for sensor data (value-based), thermal image-based data (features), and patient's medical history-based data (text-based) to identify breast abnormalities

### BRIEF DESCRIPTION OF THE DRAWING

FIG. 1 Contains Schematic of the integrated device system, the schematic shows the device with integrated mobile application for breast cancer examination that benefits patients, diagnostic centers, technical teams, and doctors.

FIG. 2 contains Brexwel Device with attached operating unit and mobile application to be used for breast cancer screening.

FIG. 3 contains details of Breast quadrants for data collection.

FIG. 4 contains workflow of the Brexwel device.

FIG. 5 shows conversion from Low resolution to High Resolution of a breast thermogram.

FIG. 6 shows the UNET Based segmentation model architecture.

FIG. 7 shows Thermograms with segmented breast region.

### DETAILED DESCRIPTION

### Materials and Methods

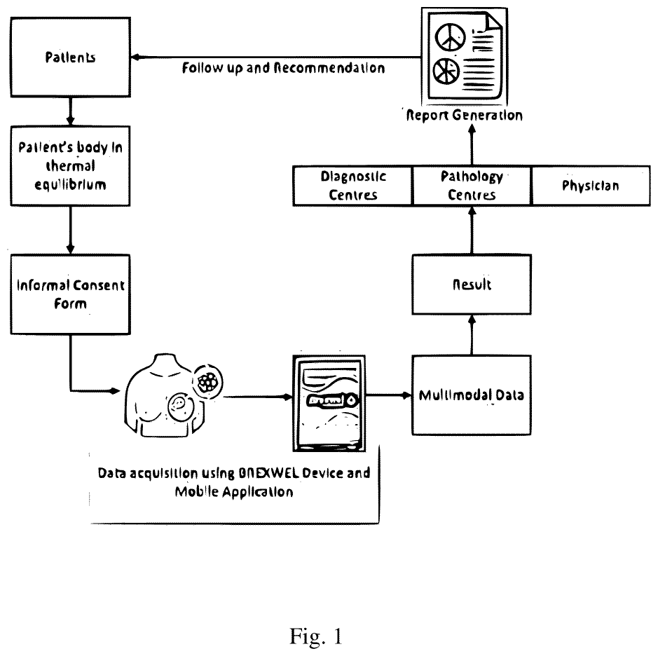

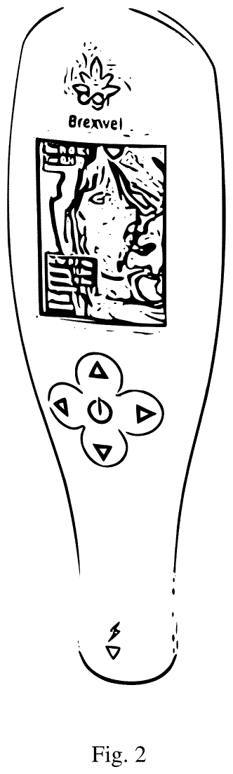

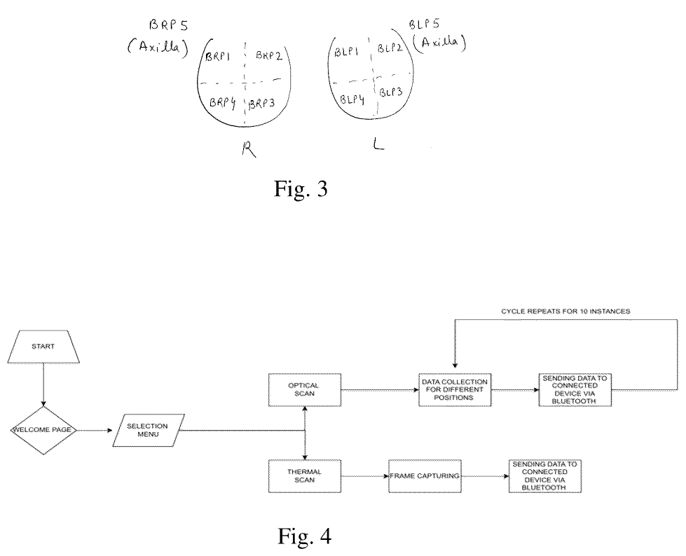

An observational prospective study was undertaken at Motilal Nehru Medical College, Prayagraj from April 2024 to December 2024. The study is funded by Startup India Seed Fund Scheme and approved by MotiLal Nehru Medical College and Associated Hospitals (Swaroop Rani Hospital, Prayagraj) Ethical Clearance Committee with IEC/MLNMC/2024/No. 26. The study is titled “Proposed Cohort Study on patients with breast lump using low-cost, portable, radiation-free, and cost-effective sensor-based breast cancer screening device”. In this study, female subjects with malignant, benign and normal breast conditions were selected to undergo the study shown in FIG. 1 and the device shown in FIG. 2 was used to take both thermal and optical data of breast quadrants. Diagram of labelling of breast quadrants is shown in FIG. 3.

The PPG sensor used in the device Max 30102 [C. P. Oximeter and H. Sensor, “Pulse Oximeter and Heart-Rate Sensor IC for Wearable Health MAX30100 Pulse Oximeter and Heart-Rate Sensor IC for Wearable Health Absolute Maximum Ratings Supply Current in Shutdown,” pp. 1-29, 2014] has ultra-low power operation (600 microamperes in measurement mode and 0.7 micro Amperes in Standby mode. The Shutdown current is 0.7 microamperes. The sample rate capability is also high 1000 samples per second for heart rate and blood oxygen level changes. There is also the integrated ambient light cancellation to adjust LED brightness according to surrounding light to reduce external interference. Package thermal resistance indicating heat dissipation to avoid overheating is 150 deg C./W. So, it can be used on body without overheating or damaging the skin.

Protocol: After ethical clearance and taking patient consent form, the process starts after creating the patient Id and entering basic details. Data will be taken in 2 different modes: Thermal and optical. For thermal mode, static breast thermogram [D. Singh, A. K. Singh, and S. Tiwari, “Early Thermographic Screening of Breast Abnormality in Women with Dense Breast by Thermal, Fractal, and Statistical Analysis,” Lect. Notes Comput. Sci. (including Subser. Lect. Notes Artif. Intell. Lect. Notes Bioinformatics), vol. 13602 LNCS, pp. 20-33, 2022, doi: 10.1007/978-3-031-19660-7_3] of female subjects will be taken by a trained medical technical professional to identify the high temperature regions for placing the sensor on breast. Well-defined breast thermogram acquisition protocols will be strictly followed for capturing images of female subjects after the approval of the ethical committee. Before the thermography and sensor data acquisition, the subject's informed consent will be taken after assuring that the identity of the subjects, associated data, and images will remain strictly confidential. Before taking static thermograms in frontal view at 1 foot. Patients will be asked to abstain from sunlight, heavy meal, or intense exercises. Based on the physician's suggestion, the subject coming for breast thermography should preferably be on her 5th-12th day and 21st day of the menstrual cycle. Images of subjects will be taken in a black chamber/cubicle with regulated temperature (which will be recorded for reference). All the personal details of both malignant and benign female subjects including name, age, sex, blood pressure, weight, menopause status, etc., and details of breast condition (symptoms), if any, and corresponding period, will be noted down. Information related to surgeries done before or family history of cancer, if any, will also be noted down for comparison. Visible distinguishing features like deformation or nipple inversion will also be recorded for correlation. Steps for thermal mode and optical mode are as follows:

### Thermal:

1. Switch ON the thermal camera through the mobile app.
2. Select the color palette as Rainbow (Refer the screenshot attached).
3. Select image mode as IR (Not MSX OR DC).
4. Select temperature range as −20 deg C. to 120 deg C.
5. Select emissivity >=0.95.
6. Select temperature units as deg C.
7. Adjust the distance to 1 foot.
8. Take the thermogram at 2 distances and upload on the cloud for each patient.

### Optical:

1. Switch ON the mobile app.
2. Pair with the Brexwel device.
3. Ensure that the distance shown is between 0.5 cm to 2.5 cm only for every position.
4. Select 1 out of 10 positions in the breast—BLP1, BLP2, BLP3, BLP4, BLP5, BRP1, BRP2, BRP3, BRP4, BRP5.
5. Connect the device.
6. Take the reading of a particular position for 30 s (TIMER).
7. Take 10-15 seconds to change the position and connect again to take data for 30 seconds.
8. Repeat for all 10 positions.
9. Show device O/P in the mobile app as H R, SpO2, and IR for every position. Only Sp02, timer, distance, and temperature displays are needed for proper positioning of the hand-held device.
10. Check the screenshot for the format of storing data.
11. Show the temperature of the particular position
12. Save in the form of Excel sheet. Export for processing.

Data will be collected through customized mobile app using thermal sensor, optical sensor, distance sensor (for positioning the device), temperature sensor. The mobile application-based device will capture and upload thermal images, sensor signal and supporting reports, to the cloud server for automatic segmentation and classification with very high accuracy and sensitivity even in the presence of noise, and improper camera angles. The device will process signal-based data, medical history-based data and thermal image-based data for cloud-based classification of suspected cases of breast cancer. The comparison with mammogram+ultrasound+MR reports will be shared to the radiologist and oncologists to validate the findings of the device. The report will be generated based on the doctor's recommendation for diagnostic test and duration of follow-up. All the regulatory compliance ISO 13485, HIPPA and ISO 27001 for Software as a Medical device (Mobile app) are taken care of to ensure device safety and maintenance of data confidentiality.

### Post Processing and Results

PATIENT FINDINGS IN BRIEF: We identified threshold of PPG signal to identify various breast conditions like malignancy, neoplastic etiology, microcalcification, fibrocystic disease, ductal ectasia as shown in Table II. There was total 18 cases of dense breast, out of which the device could identify 15 cases. Dense breasts are very common in young female population and difficult to diagnose using standard mammography. All the abnormal sensor readings correlated with the corresponding thermal hot spots (highest temperature regions) at the same quadrant of the breast region. The thermograms of 100 female subjects in frontal views were preprocessed to improve the resolution.

Table I contains comparison of the features of the Brexwel device with other similar devices/machines in the market. In this, we compared the minimum lesion size detected by the device, features like suitability in dense breast, price better than the other similar screening/diagnostic devices and machines available in the market.

Table II contains age distribution of female subjects with abnormal device readings. As seen, the highest number of females with breast abnormalities (that may lead to breast cancer after follow-up) are in the age group of 21-50.

| | | Clinical finding | | | |
|---|---|---|---|---|---|
| | Total | Abnormal | | | Patient with both |
| Age | cases | Malignant | Benign | Normal | abnormal |
| 15-20 | 8 | 0 | 7 | 1 | 8 |
| 21-30 | 30 | 0 | 26 | 4 | (Report |
| | | | | | abnormal)26 + |
| | | | | | 1(Report normal) |
| 31-40 | 19 | 4 | 10 | 5 | 15 |
| 41-50 | 14 | 4 | 8 | 2 | 10 |
| 51-60 | 5 | 1 | 2 | 2 | 4 |
| 61 & above | 2 | 0 | 2 | 0 | 2 |

Table III contains detailed distribution of female subjects with various breast problems that require continuous monitoring and follow-up. This is easily done by customized mobile application used for data collection from the device. There were 3 cases with breast abnormalities detected correctly by the device, but showed as normal in mammography report.

TABLE III
Summary of various breast conditions with
BIRADS categories identified by the device

| | | Patient | | | |
|---|---|---|---|---|---|
| | | with | | | |
| | | abnormal | | | |
| | | Brexwel | BIRADS category | Menopause | |
| S. NO. | Breast Conditions | reading | Range | Status | Diagnosis |
| 1 | Fibroadenomas (few | 25 | BIRADS II to | Pre and Post | B and M |
| | with giant) | | BIRADS IV-A | both | both |
| 2 | Micro- | 5 | BIRADS I, | Pre and Post | B and M |
| | calcification/Calcific | | BIRADS II, and | both | both |
| | foci/ | | BIRADS IV-C | | |
| | Macro-calcification | | | | |
| 3 | Neoplastic etiology | 2 | BIRADS IV and | Pre and Post | B and M |
| | | | BIRADS IV-C | both | both B |
| 4 | Fibrocystic | 1 | BIRADS II | Pre | B |
| 5 | Malignant | 9 | BIRADS II, | Pre and Post | M |
| | | | BIRADS IV-A, | both | |
| | | | BIRADS IV-C, | | |
| | | | BIRADS V and | | |
| | | | BIRADS VI | | |
| 6 | Ductal | 10 | BIRADS I, | Pre and Post | N and B |
| | dilation/Ductal | | BIRADS II, | both | both |
| | ectasia/ | | BIRADS IV-A, | | |
| | Retro areolar ductal | | and BIRADS IV-C | | |
| | prominence | | | | |
| | (macro- | | | | |
| | calcification) | | | | |
| 7 | Dense Breast | 15 | BIRADS I to | Pre and Post | N, B, and |
| | | | BIRADS IV, and | both | M |
| | | | BIRADS VI | | |

### Thermal Image Processing

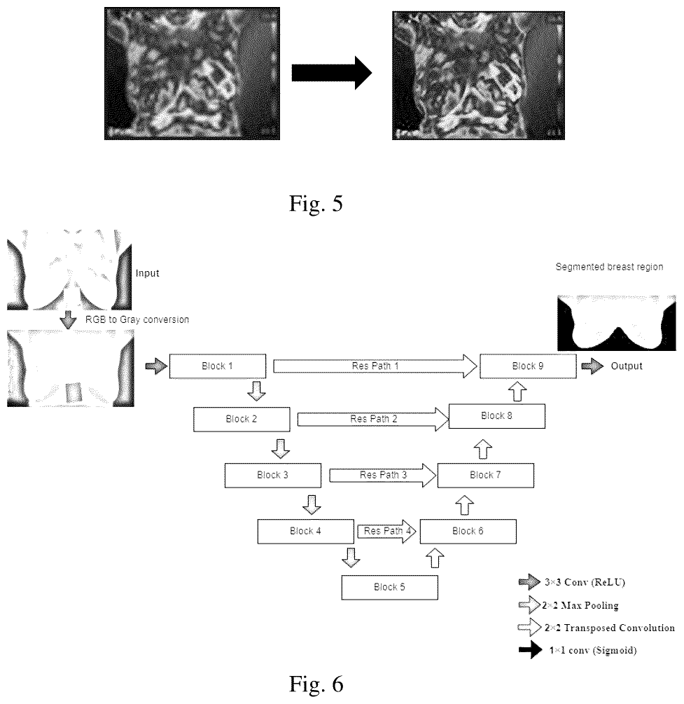

SRGAN is used for thermal image processing to improve the resolution. This Consists of a Generator to generate the images and a Discriminator to check for errors. Then the DIV2K dataset is used. Generator consists of 2 convolutional layers and Discriminator consists of 3 convolutional layers. DIV2K dataset consisting of 1000 High resolution images and 1000 low resolution images to train our model at 800 epochs. The model is tested on low quality thermal images to generate high quality images as shown in FIG. 5. We obtained the Mean Squared Error (MSE) between the images as shown in Table IV as 60.61. The PSNR is 22.79. The structural similarity Index (SSIM) between the images is 0.7835.

Table IV shows Performance of SRGAN model for conversion Low Resolution Thermal Image into High Resolution Thermal Images

| Performance measure | Obtained value |
|---|---|
| Mean Squared Error (MSE) | 60.61 |
| PSNR | 22.79 |
| Structural Similarity Index (SSIM) | 0.7835 |

We trained the segmentation model on extended dataset (720 images and their corresponding masks). Then we optimized the UNet model using Res-UNet which helps in the mitigation of the vanishing gradient problem. Number of epochs is increased to 500. We used the DBT TUJU dataset from Jadavpur University and Tripura University consisting 50 benign segmentation masks and 50 malignant segmentation masks.

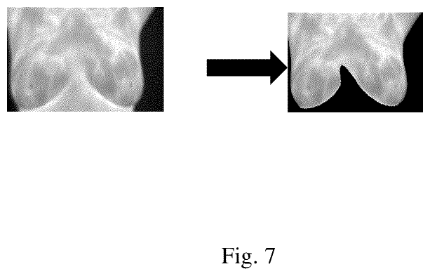

With UNET based segmentation model shown in FIG. 6 and segmented thermograms shown in FIG. 7, the Jaccard Index is 0.94017, Recall is 0.98383, Precision is 0.95429 and accuracy is 0.98660.

By applying the UNET based segmentation model, different matrices are computed such as Jaccard Index, Recall, Precision and Accuracy as mentioned in table V.

Table V shows Performance of UNET based segmentation model

| Performance of UNET based segmentation | |
|---|---|
| model | Obtained Value |
| Jaccard Index | 0.94017 |
| Recall | 0.98383 |
| Precision | 0.95429 |
| Accuracy | 0.98660 |

PPG Quality of the optical signal was improved with Feature Extraction. Extracted Feature were:
1. Amplitude Characteristics:
   Pulse Wave Amplitude
   Perfusion Index (PI)
2. Shape Characteristics:
   Number of Diastolic Peaks
   Zero-Crossing Rate
   Signal-to-Noise Ratio (SNR)

We obtained the following results Using Amplitude Characteristics
1. Logistic Regression—Accuracy: 76.03%
2. Random Forest—Accuracy: 80.00%
3. Support Vector Classifier—Accuracy: 80.00%

We obtained the following results Using Shape Characteristics
1. Logistic Regression—Accuracy: 80.00%
2. Random Forest—Accuracy: 90.00%
3. Support Vector Classifier—Accuracy: 90.00%

In this invention, we have demonstrated that females of all age groups mentioned in Table II may have breast abnormalities and in their middle age, they are more prone to have various breast disease. Proposed study is an initiative to set an alarm for all the females that they should priorities regular breast checkups including self breast examinations. As per the technical aspect, the training and testing accuracies of the used RestNet model are not as much as it was expected. The reason behind low training and testing accuracies is lower dataset, on which we are working to increase. However, accuracies and sensitives of complete analyses were 80% and 90%, respectively, which are a good result for such small data size. In future, when the data sample will increase, the performance parameters will also increase. Comparison of the complete proposed with some earlier existing work has not been mentioned as no such work has been proposed earlier. The proposed work has multiple novelties with value-added results make it more sustainable.

This invention deals with detailed optical-spectroscopy based identification of breast abnormality/lumps in Indian population using Brexwell device. The prospect of the present invention indicates that breast abnormalities can be cure if the patient is diagnosed at early stage. In the proposed work, multimodal analyses: Thermal Image based, Optical Spectroscopy based, and Patients' Medical history based, were performed. Multiple machines learning based classifiers were used to perform more accurate classification of breast abnormalities using multimodal data. A new dataset for breast abnormalities was also proposed as the data was acquired from real-time patients. A spectroscopy based device named Brexwell was proposed and an android application was designed to access the Brexwell device data. It has been seen that the accuracy for thermal image analyses was low as compared to some earlier existing literature. The reason for this was mentioned in the discussion section and the accuracy with other performance evaluation parameters can be increase by increasing the data sample. Accuracy and sensitivity for complete analyses including optical, thermal, and medical history-based data, were 80% and 93%, respectively. In future, our aim is to target more population to achieve more promising results and to spread the awareness regarding breast health care.

We claim:

1. A portable, radiation-free, and non-invasive diagnostic device for detecting abnormalities in breast tissues, comprising:
a) A sensor-based detection module configured to analyze breast lumps for micro-calcifications and neo-plastic etiology.
b) A thermal imaging unit optimized for high-resolution imaging and temperature variation analysis.
c) A processing unit with embedded machine learning algorithms trained on curated datasets for real-time abnormality classification.
d) Minimum breast lesion size identified is 4.5×1.5 mm.
e) Age group with the highest case of abnormality: 21-50 years.
f) Types of breast diseases identified: fibroadenoma, micro-calcification, neo-plastic etiology, fibrocystic, malignancy, ductal ectasia.

2. The diagnostic device as claimed in claim 1 wherein the device is integrated with data security and privacy module, comprising:
a) A user authentication and authorization mechanism to ensure controlled access.
b) End-to-end encryption of patient data during transmission and storage.

3. The diagnostic device as claimed in claim 1 wherein the processing unit is a multi-modal data processing unit configured to:
a) Collect, preprocess, and analyze thermal and optical sensor data.
b) Execute deep learning models to classify images based on temperature variations and tissue characteristics.
c) Generate diagnostic reports with anomaly detection probability metrics.

4. The diagnostic device as claimed in claim 1 wherein the device is optimized for early-stage breast abnormality detection, wherein:
a) An SRGAN model is used for improving the resolution of thermal images, with a Mean Squared Error (MSE) of 60.61, a PSNR of 22.79, and a structural similarity index (SSIM) of 0.7835.
b) A UNET-based segmentation model for thermal images achieves a Jaccard Index of 0.94017, Recall of 0.98383, Precision of 0.95429, and Accuracy of 0.98660.
c) A fusion model integrating thermal and medical-history data employs a deep neural network, achieving a training accuracy of 75% and a loss of 0.6484.
d) A support vector classifier for PPG signal quality enhancement achieves 90% accuracy.
e) Optical signal-based classification achieves 80% accuracy and 93% sensitivity.

5. The diagnostic device as claimed in claim 1 wherein the device is patient-centric mobile application which is linked to the diagnostic device, providing:
a) Secure access to diagnostic reports with interactive visualization.
b) Educational modules on breast health and early cancer detection.
c) Location-based recommendations for nearby diagnostic centers.

6. The diagnostic device as claimed in claim 1 wherein the device processing with thermal and optical image acquisition, comprising:
a) Capturing high-resolution thermal images using an infrared camera.
b) Applying noise reduction and contrast enhancement techniques to improve image quality.
c) Extracting key features related to abnormal temperature patterns for input into the machine learning classifier.

7. The diagnostic device as claimed in claim 1 wherein the device follow-up system configured to:
a) Generate customized diagnostic suggestions based on patient history and risk factors.
b) Offer reminders for follow-up screenings based on abnormality classification results.

8. The diagnostic device as claimed in claim 1 wherein the device having a noise-optimized detection mechanism operating at low-frequency ranges to ensure minimal signal interference from ambient thermal sources and enhanced reliability in varying environmental conditions.

9. The diagnostic device as claimed in claim 1 wherein the device is cost-effective and scalable design, making the device suitable for use in diagnostic centers, gynecology clinics, and rural healthcare facilities with limited resources and mobile screening camps for large-scale public health initiatives.

10. The diagnostic device as claimed in claim 1 wherein the device having multi-layered data security framework comprising Secure cloud storage with encrypted diagnostic data access and User-specific authentication protocols to protect patient confidentiality.

11. The diagnostic device as claimed in claim 1 wherein the device intuitive graphical user interface (GUI) and mobile application supporting multi-user management for healthcare professionals and customizable dashboard features for efficient data interpretation.

12. The diagnostic device as claimed in claim 1 wherein the device having multi-modal classification system integrating:
a) Value-based sensor data analysis for detecting thermal variations.
b) Feature extraction from thermal images to identify abnormal patterns.
c) Patient medical history correlation to enhance diagnostic accuracy.
